(19) United States
(12) Patent Application Publication
(10) Pub. No.: US 2005/0165389 A1
(43) Pub. Date: Jul. 28, 2005

(54) Microwave hollow organ probe

(76) Inventors: Paul Swain, London (GB); Malcolm Birch, London (GB); Jonathan Reeves, London (GB)

Correspondence Address: BACON & THOMAS, PLLC, 625 SLATERS LANE, FOURTH FLOOR, ALEXANDRIA, VA, 22314, US

(21) Appl. No.: 10/519,676

(22) PCT Filed: Jul. 8, 2003
(86) PCT No.: PCT/GB03/02948

(30) Foreign Application Priority Data
Jul. 9, 2002 (GB) 0215894.7

(51) Int. Cl. A61B 18/04

(52) U.S. Cl. 606/27; 606/33

(57) ABSTRACT

An apparatus for heat ablation of the internal wall of a hollow organ such as the oesophagus comprises a catheter having at least one internal lumen, a balloon located at the distal end of the catheter and attached to a lumen, a supply of a liquid for filling the balloon via the lumen, a tuned microwave antenna located in the region of the balloon for radiating microwave energy at a predetermined frequency to heat the balloon to a temperature suitable for heat ablation of the hollow organ wall tissue, a waveguide for supplying microwave energy to the microwave antenna, and a temperature probe to measure the temperature of the balloon. The balloon is filled from the proximal end of the catheter with a liquid having a dielectric constant of from 41 to 63 and a conductivity of from 1.0 Sm⁻¹ to 1.5 Sm⁻¹.

This invention relates to a method and apparatus for heat ablation of the internal wall of hollow organs.

Microwave hyperthermia treatments have for many years been used for treatment of cancers. It is known that the raising of the temperature of cells to above about 43° to 45° C. for sufficient time causes necrosis, and temperatures below about 41.5° C. generally do not affect cells. External hyperthermia treatments for the treatment of Barrett's oesophagus, a pre-malignant condition of the oesophagus cannot be used because of the anatomical site of the oesophagus. However, intracavitary hyperthermia allows the applicator to touch the lesion directly, with almost all of the microwave energy being absorbed by the area surrounding the applicator, sparing the normal tissues from excessive exposure to heat. Work to improve the heating pattern of an oesophageal applicator is described in Int. J. Hyperthermia 1991 Vol. 7 No. 4 pp 577-586 Liu et al. This describes a microwave applicator shrouded in a plastic tube of 1 cm diameter. This type of system is incapable of treating the entire affected area of the oesophagus at the same time.

Applicators of the type described in the Liu article are common in microwave hyperthermia. In U.S. Pat. No. 5,843,144, such an applicator is used in the treatment of the prostate. The problem with applicators of this kind is that it is difficult to control the heat flow from the microwave antenna. As it is difficult to direct the heat to the area requiring treatment accurately, damage to healthy tissue could result.

U.S. Pat. No. 5,222,938 employs a thermally conductive liquid medium to assist in controlling the heat flow from a heating means in the gall bladder. The thermally conductive liquid is not contained within a balloon but rather is in direct contact with the wall of the gall bladder, and is constrained by inflatable cuffs which hold the heating means in position during the treatment. The heating means is typically “a resistive heater or a radiating block heated by laser energy or the like”. However, the use of a microwave emitter is mentioned. Examples suggested for the thermally conductive liquid are “water, saline, contrast medium, physiological irrigating solution and the like”. There is no mention of the dielectric constant or conductivity of the solution, nor any suggestion that any particular liquid is desirably used to achieve improved heat flow to the tissue needing treatment.

In U.S. Pat. No. 5,057,106, a microwave antenna is positioned inside a balloon for use in microwave balloon angioplasty. The balloon surface is coated with a lossy material which will allow absorption of microwave energy. The balloon contains a low loss material which is designed to minimise the heating within the balloon. In the use of a system of this type in, for example, the oesophagus, the required large distance between the antenna and lumen wall required, and the change in dielectric value between the balloon contents and the surrounding tissue would produce reflected microwave power. This would mean irregular heating of the lumen wall and heating of the waveguide, resulting in failure to ensure that the entire affected area of the hollow organ is treated at the correct temperature and in damage to healthy tissue along the route of insertion.

It is therefore desirable to produce an apparatus which is capable of treating the whole of the affected area of the hollow organ with minimal risk to the surrounding healthy tissue, either from misdirected heat from the microwave antenna or from the waveguide heating up in a poorly matched system.

This kind of treatment is particularly suited to the oesophagus. The oesophagus can be affected by Barrett's oesophagus, as well as cancer of the oesophagus or areas of dysplasia. Current experimental approaches to treating Barrett's oesophagus are Photodynamic therapy (PDT), direct laser action and microwave hyperthermia.

In PDT, a photosensitising drug is given which accumulates in the malignant tissue. Irradiating the target tissue with a laser activates the drug. The activated drug causes tissue destruction, probably by the production of singlet oxygen. The problems associated with the administration of exogenous photosensitisers are low selectivity, accumulation in malignant tissues, and skin sensitivity for up to several months. These photosensitisers also result in the formation of strictures in up to 45% of patients. To overcome these problems endogenous photosensitisers have been developed. Strictures have not been reported after this improved treatment. However, residual Barrett's epithelium has been found in a large number of patients after follow up.

Direct laser action may produce vaporisation with immediate destruction whenever tissue temperature exceeds 100° C. Coagulation necrosis of the tumour with delayed slough occurs between 60° C. and 90° C. Lasers offer the advantage of rapid thermal destruction of cancerous and pre-malignant tissue but it is not possible to control the tissue temperature. The endoscopist must gauge the effect by visual cues and experience. Unfortunately, lasers treat a limited area with each firing. A point by point therapy is performed. Through a striping motion it is possible to carry the point ablation over larger areas. There is an uneven quality to this type of treatment, which cannot be avoided. Laser ablation therapy is laborious to perform and commonly takes up to 8 sessions. The system also depends on the ‘freehand’ endoscopic control by the clinician.

Accordingly, in a first aspect, the present invention provides an apparatus for heat ablation of the internal wall of a hollow organ. The apparatus comprises a catheter having proximal and distal ends, and having at least one internal lumen. A balloon is located at the distal end of the catheter and attached to a said lumen, whereby the balloon may be filled with a liquid from the proximal end of the catheter. A tuned microwave antenna is located in the region of the balloon for radiating microwave energy at a predetermined frequency to heat the balloon to a temperature suitable for heat ablation of the hollow organ wall tissue. A waveguide is attached to the microwave antenna. The wave guide supplies microwave energy to the microwave antenna. A temperature probe is also provided to measure the temperature of the balloon. A supply of a liquid is provided for filling the balloon via the said lumen. The liquid has a dielectric constant of from 41 to 63 and a conductivity of from 1.0 Sm⁻¹ to 1.5 Sm⁻¹ at said frequency and 50° C. High water content tissue, which is the type of tissue to be treated, has a dielectric constant of 53 at a microwave frequency of 433 MHz, and a dielectric constant of 51 at 915 MHz. It also has a conductivity of 1.18 Sm⁻¹ at 433 MHz and a conductivity of 1.28 Sm⁻¹ at 915 MHz. The dielectric constant of the liquid employed in the apparatus is preferably within 20% of the average of the dielectric constant values at the two frequencies and the conductivity of the liquid employed is preferably within 20% of the average of the conductivity values at the two frequencies. The matching of the dielectric constant and conductivity of the liquid used with the dielectric constant and conductivity of the high water content tissue allows improved matching of the microwave antenna and the waveguide, thus reducing heating of the waveguide.

The dielectric constant of the liquid employed in the apparatus is preferably within 10% of the average value of the dielectric constant for high water content tissue at 433 MHz and 915 MHz. Therefore the dielectric constant is preferably from 47 to 57. The conductivity of the liquid employed in the apparatus is preferably within 10% of the average value of the conductivity for high water content tissue at 433 MHz and 915 MHz. Therefore the conductivity is preferably from 1.1 to 1.35 Sm⁻¹.

The apparatus is particularly suitable for treating the oesophagus.

In order for the balloon to fit the internal dimensions of the oesophagus, it is preferable that the balloon has a normal inflation diameter of from 16 to 22 mm. The normal inflation diameter of the balloon is the diameter to which the balloon is designed to be inflated to i.e. that it is readily inflated to without the application of excessive pressure.

It is preferable that neither the balloon nor the temperature probe have any metal containing components as the presence of metal will affect the microwave heating pattern, resulting in uneven heating.

The use of at least one optical fibre extending from the distal end to the proximal end of the tube as the temperature probe is therefore preferable.

A feedback system is preferably employed to ensure that the area of the hollow organ requiring treatment is heated to and maintained at the correct temperature. Such a system operates by adjusting the microwave power supplied to the microwave antenna in accordance with the temperature sensed by the temperature probe. This ensures that excessive heating of the internal body will not occur.

In order to assist with insertion of the balloon into the desired location, a guidewire may be attached to the balloon. Depending on the material used to make the guidewire, the guidewire can be removed prior to the procedure, or if it will not affect the microwave heating pattern, it may remain in place during the procedure.

### DESCRIPTION OF PREFERRED EMBODIMENTS

The invention will be further described with reference to the preferred embodiments shown in the accompanying drawings, in which:

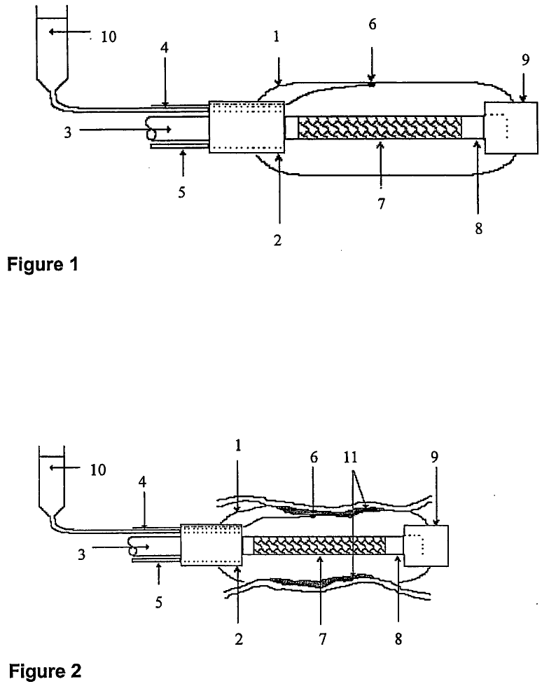

FIG. 1 shows a schematic of the balloon of the preferred embodiment.

FIG. 2 shows a schematic of the balloon of FIG. 1 in position in the oesophagus.

FIG. 1 shows a balloon 1 attached and sealed to a support 2 made of a plastics material. The support 2 has four internal channels passing into the balloon namely a central channel for a waveguide 3 and three smaller channels for a liquid inlet tube 4, an air outlet tube 5 and an optical fibre 6. The waveguide 3 is connected to one end of an antenna 7. The other end of the antenna 7 is connected via a solid tip 8 to a former 9 to centralise the antenna 7. The aveguide 3, the optical fibre 6 and the inlet and outlet tubes 4 and 5 are approximately 0.5 m in length i.e. of ufficient length to extend out of the body. Outlet tube 5 is connected to a bleed valve (not shown). The proximal end of the waveguide 3 is connected to a microwave generator (not shown). Inlet tube 4 is connected to a supply of a liquid 10 having a dielectric constant of from 47 to 57 and a conductivity of from 1.1 to 1.35 Sm⁻¹. The liquid 10 consists of deionised water, with a suitable sugar added to alter the dielectric constant and NaCl added to increase the conductivity. The liquid 10 also includes a hydroxycellulose-based viscosity modifier, which provides a jelly-like consistency, and helps to reduce convection currents.

FIG. 2 shows the balloon 1 positioned in the oesophagus adjacent to a region 11 to be treated. The balloon 1 is inflated by the liquid 10.

In use, the balloon 1 is inserted into the oesophagus and positioned adjacent to the region 11 to be treated. Liquid 9 is injected into the balloon 1 through inlet tube 4. The bleed valve (not shown) attached to the outlet tube 5 allows air bubbles to be removed from the balloon 1. The balloon 1 is inflated by liquid 10 until it reaches its normal inflation diameter. The region to be treated 11 is in contact with the surface of balloon 1.

The region 11 to be treated is treated by heating it to tumourcidal temperatures. Microwave power is supplied to the antenna 7 using the microwave generator (not shown). The liquid 10 in the balloon 1 is heated by microwaves emitted by antenna 7. The liquid 10 heats the balloon 1. The surface of the balloon 1 heats the region 11 to be treated. The temperature of the balloon 1 is measured using the optical fibre 6. A feedback system (not shown) uses the temperature sensed by the optical fibre 6 to correct the amount of microwave power emitted by the microwave generator and provided to the antenna 7 in order to ensure that the region of the oesophagus to be treated 11 is heated to and maintained at the correct temperature. After use, the balloon 1 is deflated using the inlet tube 4.

The preferred embodiment of the invention described above and shown in FIG. 1 and FIG. 2 has several advantages over the methods of heat ablation of the oesophagus and the methods of microwave hyperthermia in the prior art.

The liquid used to fill the balloon has the same dielectric constant and conductivity values as those of oesophageal tissue. The use of this liquid allows improved matching of the microwave antenna and the waveguide due to the identical nature of the liquid surrounding the microwave antenna and the tissue outside the balloon. The termination impedence of the waveguide is closely matched to its characteristic impedence, thereby avoiding reflection of microwave power back into the waveguide. Such reflected microwave power would cause the waveguide to become hot, resulting in heating of and possible damage to the surrounding tissue.

The use of the liquid having the same dielectric constant and conductivity as oesophageal tissue allows the heat transfer characteristics from the liquid in the balloon to the oesophagus wall to be more readily predicted than if a liquid with different dielectric constant and conductivity properties were used.

During the construction of a microwave antenna for use in apparatus according to the invention, it is necessary to check the matching of the antenna to the waveguide experimentally. This is preferably carried out whilst the antenna is inserted in a so-called muscle equivalent phantom i.e. a liquid having the same dielectric constant and conductivity as tissue. It is therefore a further benefit of the method of the invention that the antenna will perform identically in a balloon filled with the liquid as it did when tested in the tissue equivalent phantom.

The inflation of the balloon minimises any air gaps between the balloon wall and the tissue requiring treatment. Air gaps will affect the composite dielectric constant value of the volume between the microwave antenna and the tissue requiring treatment. This has a dramatic effect on the thermal distribution from the antenna and the matching of the system.

The balloon is also able to take up the shape of the oesophageal region and will flatten the mucosal folds, preventing any areas from being shielded. The balloon will therefore directly touch the area of tissue to be treated. It is therefore possible to deliver energy over the whole of the region requiring treatment, destroying the full thickness of the mucosa without damaging the underlying muscle.

The inflated balloon may be held stationary within the oesophageal lumen throughout the treatment without the need for repositioning.

The extent of tissue requiring treatment will differ from patient to patient, as will the size of the oesophagus. It is therefore possible to create a discrete set of balloons with varying lengths and diameters for customised clinical treatment.

Whilst the invention has been described with reference to the illustrated preferred embodiments, it is to be appreciated that many modifications and variations are possible within the scope of the invention.

1. Apparatus for heat ablation of the internal wall of an oesophagus, which apparatus comprises;
a catheter having proximal and distal ends, and having at least one internal lumen;
a balloon located at the distal end of the catheter and attached to a said lumen,
whereby the balloon may be filled with a liquid from the proximal end of the catheter;
a supply of a liquid for filling the balloon via the said lumen;
a tuned microwave antenna located in the region of the balloon for radiating microwave energy at a predetermined frequency to heat the balloon to a temperature suitable for heat ablation of the hollow organ wall tissue;
a waveguide for supplying microwave energy to the microwave antenna;
a former to centralize the antenna; and
a temperature probe to measure the temperature of the balloon;
wherein the liquid has a dielectric constant of from 41 to 63 and a conductivity of from 1.0 Sm⁻¹ to 1.5 Sm⁻¹ at said frequency and 50° C.

2. Apparatus as claimed in claim 1 wherein the liquid has a dielectric constant of from 47 to 57 at said frequency and 50° C.

3. Apparatus as claimed in claim 1 wherein the liquid has a conductivity of from 1.1 to 1.35 Sm⁻¹ at said frequency and 50° C.

4. (canceled)

5. Apparatus as claimed in claim 1 wherein the balloon has a normal inflation diameter of from 16 to 22 mm.

6. Apparatus as claimed in claim 1 wherein the temperature probe and the balloon contain no metal.

7. Apparatus as claimed in claim 6, wherein the temperature probe comprises at least one optical fibre extending from the distal end to the proximal end of the tube.

8. (canceled)

9. A process for heat ablation of the internal wall of an oesophagus of a patient, comprising the steps of; providing a catheter having proximal and distal ends and having at least one internal lumen wherein a balloon is located at the distal end of the catheter and is connected to a said lumen, the balloon surrounding a tuned microwave antenna and a temperature probe and wherein a waveguide for supplying microwave energy at a predetemmined frequency to the microwave antenna is connected to the microwave antenna;
inserting the distal end of the catheter into the hollow organ;
positioning the catheter such that the balloon is adjacent to the area of the hollow organ requiring heat ablation;
filling the balloon via the said lumen with a liquid having a dielectric constant of from 47 to 57 and a conductivity of from 1.0 Sm⁻¹ to 1.5 Sr⁻¹ at said frequency and 50° C.;
supplying microwave energy via the waveguide to the microwave antenna to heat the balloon.

10. A process as claimed in claim 9 comprising the further 35 steps of; providing a means for controlling the power supplied to the microwave antenna in dependence upon the temperature sensed by the temperature probe; and
controlling the power supplied to the microwave antenna to ensure heat ablation of the oesophagus of the patient.

11. (canceled)

12. Apparatus as claimed in claim 1, including means for controlling the power supplied to the microwave antenna in dependence upon the temperature sensed by the temperature probe.
